(19) United States
(12) Patent Application Publication
(10) Pub. No.: US 2007/0298005 A1
(43) Pub. Date: Dec. 27, 2007

(54) INJECTABLE COMPOSITION FOR TREATMENT OF SKIN DEFECTS OR DEFORMATIONS

(76) Inventor: Marie-Josee Thibault, Encinitas, CA (US)

Correspondence Address: WILSON SONSINI GOODRICH & ROSATI, 650 PAGE MILL ROAD, PALO ALTO, CA, 94304-1050, US

(21) Appl. No.: 11/766,629

(22) Filed: Jun. 21, 2007

Related U.S. Application Data
(60) Provisional application No. 60/805,563, filed on Jun. 22, 2006.
(60) Provisional application No. 60/866,790, filed on Nov. 21, 2006.

(51) Int. Cl. A61K 8/97; A61K 31/275; A61K 36/47; A61K 8/37

(52) U.S. Cl. 424/70.31; 424/74; 424/731; 514/524

(57) ABSTRACT

Provided are injectable compositions for correction of skin contour defects or deformations. Also provided are methods of use and manufacturing of compositions for correction of skin contour defects or deformations.

### CROSS-REFERENCE

This application claims the benefit of U.S. provisional applications Ser. Nos. 60/805,563 filed Jun. 22, 2006, and 60/866,790 filed on Nov. 21, 2006, which are both incorporated by reference in their entireties.

### FIELD OF THE INVENTION

The invention relates to the use of compounds and derivatives from the family of alkylene glycols, including ethylene glycols, for the treatment of skin contour defects or deformations and to compositions thereof.

### BACKGROUND OF THE INVENTION

Skin contour defects or deformations in the form of scarring, wrinkles, sagging or folded skin, and thinning of lips are a common and inevitable problem in virtually all people. The primary cause of such deformations is aging, but such deformations can be initiated or exacerbated by other events such as physical trauma, illness, or even unstoppable events such as puberty which often brings acne scarring.

Skin consists of four main compartments (the epidermis, the papillary dermis, the reticular dermis, and the hypodermis), and the visible topography is controlled by the architecture and tensile interactions between the four compartments. Skin contour deformations, such as wrinkles, are due to changes within the four compartments as the skin ages. There is progressive loss of thickness as well as dermal volume throughout all compartments, with loss of the fat volume and thinning of the connective tissue strands most prominent in the hypodermis. Additional contributors to skin contour deformations include further rigidification of the epidermis and papillary dermis, decreased elasticity and increased extensibility of both the reticular dermis and hypodermis, and increased sagging, laxity and folding capacity of the hypodermis.

Wrinkles, as a type of skin contour defect or deformation, are further divided into 4 types in addition to lip thinning. Type 1 wrinkles appear as fine wrinkles anywhere on the body and are characteristic of the aging process. Type 2 wrinkles appear as deep wrinkles mainly on sun-exposed areas and are initially reducible but progress to permanent wrinkles. Type 3 wrinkles appear deeper and exhibit more folding, and their appearance and placement coincides with facial muscle use. Finally, type 4 wrinkles appear as deep wrinkles and folds, often compounded by sagging of skin, and are attributed to changes in the three-dimensional topography of the face related to aging. Also type 4 wrinkles but with different etiology, “sleep lines” are due to repetitive pressure applied by pillows on specific areas of skin due to sleep patterns and habits. Finally, thinning of the lip area results in a loss of natural thickness such that lip height and volume decrease as well as the appearance of wrinkles, especially at the corners of the mouth. Often associated with lip thinning, chin atrophy and associated depressions can appear below the lips themselves.

In additional to wrinkling, scarring of the skin, as a type of skin contour defect or deformation, is a significant problem within the world's population. Scarring is a permanent alteration in the topography of the skin as the result of some trauma. Scars are divided into a variety of categories depending upon the origin of the trauma and/or the location. Scarring is not age-related like most wrinkling, and the presence of scars typically implies a process of compromised wound healing combined with various degrees of necrosis.

Prior therapies for skin contour corrections such as wrinkles, lip thinning, and scarring include surgery, ablative remodeling procedures, fillers, and steroid injections for the reduction of hypertrophic or keloidal scars. These solutions have many undesirable and potential complications including long recovery periods and unforeseen reactions to general surgery, incomplete correction as with ablative therapies (e.g., chemical peels and laser treatments), and paralysis and atrophy of muscles (e.g., associated with Botox™ injection). In view of such concerns, soft tissue augmentation with fillers has been identified as popular option for corrections of skin deformations and contour corrections. While fillers reportedly have longevities of 6 months to up to 2 years, numerous adverse reactions have been reported. Repeated instances of hypersensitivity and inflammatory reactions, necrosis, infection, cystic reactions, systemic complaints, foreign body reactions, acneiform reactions, granulomas, and lumping, migration, displacement or extrusion of the implants have been reported. Further, many current fillers result in lumpy or unattractive areas, often further complicating the original skin deformation or simply replacing one problem with another. Examples of such fillers include bovine collagen and various polymers such as polyvinylpyrrolidone, alginates, silicone, and lactones.

Thus, there is a need for skin contour corrections which minimize or eliminate recovery time. There is also a need for skin contour corrections that have few side-effects or fewer side-effects than current therapies. There is yet another need for skin contour corrections that do not wound the skin surface. Similarly, there is a need for skin contour corrections that offer smooth, natural, three-dimensional and visually pleasing correction.

### SUMMARY OF THE INVENTION

Described herein are compositions and methods for treating skin contour defects or deformations comprising at least one hydrophilic polymer, alkoxylated oil, polyalkylated glycol, or combinations thereof, and a physiologically acceptable solvent. Optionally, such compositions may further comprise an anesthetic, preservative, and/or tonicity agent.

### DETAILED DESCRIPTION OF THE INVENTION

The present invention provides injectable compositions of hydrophilic polymers, alkoxylated oils, and/or polyalkylated glycols for correction of skin contour defects or deformations, as well as methods of use and manufacturing of such compositions. An aspect of the invention is drawn to pharmaceutically acceptable, injectable compositions comprising at least one hydrophilic polymer; pharmaceutically acceptable, injectable compositions comprising at least one alkoxylated oil; pharmaceutically acceptable, injectable compositions comprising at least one polyalkylene glycol; and pharmaceutically acceptable, injectable compositions comprising a combination of at least one hydrophilic polymer, alkoxylated oil, or polyalkylated glycol.

In an embodiment, the present pharmaceutically acceptable, injectable compositions comprise at least one ethoxylated castor oil and a physiologically acceptable solvent. In an embodiment, the ethoxylated castor oil is Cremophor ELP (sold by BASF). In yet another embodiment, the present compositions comprise at least one soft tissue filler. In yet another embodiment, the present compositions comprises at least one local anesthetic.

A further aspect of the invention is a method of correcting skin contour defects or deformations by injecting said compositions into the skin. In another embodiment of the invention, including the aforementioned embodiments and aspects, the composition is injected into at least one compartment of the skin. In a further embodiment, including the aforementioned embodiments and aspects, the composition is injected into the reticular dermis and the papillary dermis. In yet another embodiment, including the aforementioned embodiments and aspects, the composition is injected first into the reticular dermis followed by injection into the papillary dermis. In one embodiment of the invention, including the aforementioned embodiments and aspects, the injectable composition is used to treat wrinkles, such as to reduce the appearance of wrinkles and/or fine lines. In another embodiment, including the aforementioned embodiments and aspects, the injectable composition is used to treat scarring, such as to reduce the appearance of scarred tissue. In a further embodiment, including the aforementioned embodiments and aspects, the injectable composition is used to treat lip thinning, such as to enhance the appearance of the lips, facilitate the appearance of a puffier pout, smooth lines around the mouth. In yet a further embodiment, the injectable composition is used as a regenerator to increase the endogenous renewal of the skin.

Another aspect of the invention is a method of manufacturing said compositions for the correction of skin contour defects or deformations.

The term “polyalkylated” by itself or as part of another substituent, means, unless otherwise stated, more than one straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkoxylated” is used in its conventional sense, and refers to those alkyl groups attached to the remainder of the molecule via an oxygen atom.

The term “ethoxylated” refers to an ethyl group attached to the remainder of the molecule via an oxygen atom.

The term “polyoxyethylene” refers to more than one ethyl group that is attached to the remainder of the molecule via an oxygen atom.

As used herein, the term “polyalkylated glycol” or “polyoxyethylene glycol” refers to polyethylene glycol (poly(ethylene glycol)), polypropylene glycol, polybutylene glycol, and derivatives thereof. The term encompasses both linear and branched polymers and average molecular weights of between about 0.1 kDa and about 100 kDa. Other exemplary embodiments are listed, for example, in commercial supplier catalogs, such as Shearwater Corporation's catalog “Polyethylene Glycol and Derivatives for Biomedical Applications” (2001).

The term “hydrophilic polymer” as used herein refers to any suitable polymer that is soluble in aqueous solvents. Suitable polymers include, but are not limited to, polyethylene glycol, polyethylene glycol propionaldehyde, mono C₁-C₁₀ alkoxy or aryloxy derivatives thereof (e.g., as described in U.S. Pat. No. 5,252,714 which is incorporated by reference herein), monoethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, starch and starch derivatives, polypeptides, polyalkylene glycol and derivatives thereof, copolymers of polyalkylene glycols and derivatives thereof, polyvinyl ethyl ethers, and alpha-beta-poly(2-hydroxyethyl)-DL-aspartamide, and the like, or mixtures thereof.

### Compositions

Injectable compositions for treatment of skin contour defects or deformations comprise hydrophilic polymers, alkoxylated oils, and/or polyalkylated glycols. These polymers may be modified to further improve their usefulness for the treatment of skin defects or deformations. In an embodiment, the injectable compositions comprise at least one ethoxylated castor oil, such as, but not limited to, Cremophor ELP. Cremophor ELP (chemical name polyoxyethyleneglycerol triricinoleate) is a compound obtained by the reaction of castor oil and ethylene oxide in a molar ratio of 1:35. Purification of the reaction product results in a soluble liquid. Cremophor ELP generates little sensitivity or inflammatory reactions when administered, and Cremophor ELP is biodegradable and generally safely metabolized by the body. Cremophor ELP has been reported as a solubilizing agent for parenteral dosage forms, particularly with oncology drugs. As described herein, ethoxylated castor oil, such as Cremaphor ELP comprises one embodiment of compounds useful in the present invention. Other ethoxylated castor oil compositions besides Cremophor ELP may be used, e.g., by reaction of castor oil and ethylene oxide in a ratio of between about 1:15 and about 1:70, between about 1:20 and about 1:60, between about 1:20 and about 1:50, between about 1:25 and about 1:55, and between about 1:30 and about 1:45. Described herein in some embodiments of the invention are novel uses for hydrophilic polymers, alkoxylated oils, polyaklylated glycols, ethoxylaged castor oil, and Cremophor ELP, and novel compositions comprising such components.

In manufacturing the present injectable compositions, hydrophilic polymers, alkoxylated oils, and/or polyalkylated glycols are solubilized in a physiologically acceptable solvent. A variety of physiologically acceptable solvents may be used including, but not limited to, water, saline and other various salt and/or buffering solutions. Further physiologically acceptable solvents for parenteral administration include a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a further, pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. The physiologically acceptable solvent used can be easily determined by one of skill in the art.

The present injectable composition comprise from about 0.5% to about 50% weight/weight of at least one hydrophilic polymer, alkoxylated oil, and/or polyalkylated glycol. Preferably, the injectable compositions comprise an alkoxylated oil, in particular, Cremophor ELP. The specific concentration of the hydrophilic polymer, alkoxylated oil, and/or polyalkylated glycol may be determined by, but is not limited to, assessment of the skin contour defect or deformation to be treated, area of skin to be treated, size of skin contour defect or deformation, and other physiological parameters identified by treating physician or health care professional. Solubilized compositions comprising at least one hydrophilic polymer, alkoxylated oil, and/or polyalkylated glycol in a physiologically acceptable solvent are sterilized prior to use. Exemplary sterilization techniques include autoclave sterilization, however alternative sterilization techniques may be applied and are easily identified by one of skill in the art. Following sterilization, the solution may experience some separation while cooling. Mixing or shaking the composition occasionally as it cools will facilitate re-distribution or re-solubilization of the hydrophilic polymer, alkoxylated oil, and/or polyalkylated glycol in the chosen solvent.

Anesthetics may also be included in the injectable composition. If an anesthetic is desired in the composition, a sterile, injectable solution of the chosen anesthetic may be added, preferably following sterilization of the solution. Lidocaine HCl can be included in the composition in concentrations ranging from about 0.1% to about 10% w/w. Additional anesthetics which can be included in the present compositions are benzocaine, mepivacaine, ropivacaine, bupivacaine, prilocalne, procaine, chloroprocaine, and similar compounds known in the art. The choice, amount and administration of an anesthetic can be easily identified by one of skill in the art.

Similarly, preservatives and/or tonicity agents may be added to the solution for prolonging the shelf-life and further promoting the biological tolerance and comfort of the injected solution. Suitable preservatives for inclusion in the solution include, but are not limited to, esters of para-hydroxybenzoic acid (e.g., methyl, ethyl, propyl and butyl esters, or mixtures of them), chlorocresol and the like: propyl gallate; sorbic acid and its sodium and potassium salts; propionic acid and its calcium and sodium salts; 6-acetoxy-2,4-dimethyl-m-dioxane; 2-bromo-2-nitropropane-1,3-diol; salicylanilides such as dibromosalicylanilide and tribromosalicylamilide, the cis isomer of 1-(3-chloroallyl)-3,5,7-triaza-1-azanidadamantane chloride; hexachlorophene; sodium benzoate; chelating agents such as ethylene diaminetetraacetic acid (EDTA), citric acid, and their alkali metal salts. Suitable tonicity adjustment agents include, but are not limited to, physiologically acceptable inorganic chlorides, e.g. sodium chloride, dextrose, lactose, mannitol and the like.

In an embodiment, the present injectable compositions comprise Cremophor ELP in which the Cremophor ELP is utilized as a soft tissue filler. The preferred concentration of Cremophor ELP in this embodiment is about 40% w/w. In some embodiments, a physiologically acceptable solvent with low hypertonicity is used, for example, water. Any of such combinations of a hydrophilic polymer, such as Cremophor ELP, are mixed via conventional mixing techniques. The Cremophor ELP mixture is preferably autoclaved in a sterile container at 121 degrees centigrade for 30 minutes, and then shaken at least one time while cooling to room temperature. A solution of injectable lidocaine HCl, or any other acceptable local anesthetic, is added to the Cremophor ELP solution and mixed. The final concentration of lidocaine HCl may be up to about 5% w/w. In some embodiments, the final concentration of lidocaine HCl is from about 0.5% to about 1.5% w/w. In other embodiments, the concentration is about 1% w/w.

In an embodiment, the present injectable compositions comprise at least one alkoxylated oil, such as an ethoxylated castor oil. Exemplary ethoxylated castor oils which may be used in the present composition include, but are not limited to, PEG-10 Castor oil, PEG-100 Castor oil, PEG-1 Castor oil, PEG-15 Castor oil, PEG-2 Castor oil, PEG-20 Castor oil, PEG-200 Castor oil, PEG-25 Castor oil, PEG-26 Castor oil, PEG-3 Castor oil, PEG-30 Castor oil, PEG-33 Castor oil, PEG-35 Castor oil, PEG-36 Castor oil, PEG-4 Castor oil, PEG-40 Castor oil, PEG-5 Castor oil, PEG-50 Castor oil, PEG-54 Castor oil, PEG-55 Castor oil, PEG-60 Castor oil, PEG-8 Castor oil, PEG-9 Castor oil, Polyethoxylated castor oil, Polyethylene glycol (100) castor oil, Polyethylene glycol (11) castor oil, Polyethylene glycol (15) castor oil, Polyethylene glycol (25) castor oil, Polyethylene glycol (26) castor oil, Polyethylene glycol (3) castor oil, Polyethylene glycol (30) castor oil, Polyethylene glycol (33) castor oil, Polyethylene glycol (35) castor oil, Polyethylene glycol (5) castor oil, Polyethylene glycol (50) castor oil, Polyethylene glycol (54) castor oil, Polyethylene glycol (55) castor oil, Polyethylene glycol (60) castor oil, Polyethylene glycol 1000 castor oil, Polyethylene glycol 1800 castor oil, Polyethylene glycol 200 castor oil, Polyethylene glycol 2000 castor oil, Polyethylene glycol 400 castor oil, Polyethylene glycol 450 castor oil, Polyethylene glycol 500 castor oil, Polyoxyethylene (10) castor oil, Polyoxyethylene (100) castor oil, Polyoxyethylene (11) castor oil, Polyoxyethylene (15) castor oil, Polyoxyethylene (2) castor oil, Polyoxyethylene (20) castor oil, Polyoxyethylene (200) castor oil, Polyoxyethylene (25) castor oil, Polyoxyethylene (26) castor oil, Polyoxyethylene (3) castor oil, Polyoxyethylene (30) castor oil, Polyoxyethylene (33) castor oil, Polyoxyethylene (35) castor oil, Polyoxyethylene (36) castor oil, Polyoxyethylene (4) castor oil, Polyoxyethylene (40) castor oil, Polyoxyethylene (5) castor oil, Polyoxyethylene (50) castor oil, Polyoxyethylene (54) castor oil, Polyoxyethylene (55) castor oil, Polyoxyethylene (60) castor oil, Polyoxyethylene (8) castor oil, Polyoxyethylene (9) castor oil, and the like.

Exemplary ethoxylated castor oils that can be included in the present compositions are those sold or used under the following names: AQ 250, Actinol CS 40, Alkamuls EL 620, Alkamuls OR 36, Alkasurf CO 10, Alkasurf CO 15, Alkasurf CO 25B, Alkasurf CO 40, Arlatone 285, Arlatone 650, Atlas G 1281, Atlas G 1300, Bio-Soft HR 40, Bio-Soft HR 55, Castor oil polyoxyethylene ether, Castor oil ethoxylated, Castor oil ethylene glycol polymer, Castor oil ethylene oxide reaction product, Castor oil polyethoxylated, Cemulsol BR, Chemax CO, Cremaphor, Cremophor EL, EO 90, Emanon CH 80, Emulan EL, Emulan ELP, Emulphor EL 620, Emulphor EL 620P, Emulphor EL 719, Emulphor EL 749, Emulphor EL 980, Emulphor EL-620, Emulpon EL 40, Emulsogen EL, Emulsogen EL 400, Ethofor RO 40, Ethox CO 25, Ethox CO 30, Ethoxylated castor oil, Etocas 35, Etocas 40, Etocas 5, Eumulgin HRE 40, Eumulgin RO 40, Eumulgin RT 40, G 1300, HCO 505, Hairikku AQ 250, Heliwet EO 33, Hextoxide C 15, Hextoxide C 25, Polyoxyethylated castor oil, and the like.

Compositions presented herein can optionally be combined with other tissue fillers known in the art including, but not limited to, collagen from bovine skin (such as those marketed, for example, as Zyderm® and/or Zyplast®); autologous collagen from human source (such as those marketed, for example, as Cosmoderm® and/or Cosmoplast®); collagen fibers prepared from patient tissue (such as those marketed, for example, as Autologen®); collagen fibers prepared from cadaveric human dermis (such as those marketed, for example, as Dermalogen® and/or Cymetra®); cultured autologous fibroblasts from patients (such as those marketed, for example, as Isolagen®); collagenous tissue from human donor fasica (such as those marketed, for example, as Alloderm®); non-animal derived hyaluronic acid (such as those marketed, for example, as Restylane® and/or Perlane®); hyaluronic acid from rooster combs of domestic fowl (such as those marketed, for example, as Hylan-B®); 75% collagen, 25% PMMA Plexiglas spheres (such as those marketed, for example, as Artecoll®); calcium hydroxyapatite microspheres (such as those marketed, for example, as Radiesse®); expanded polytetrafluoroethylene prepared from Teflon (such as those marketed, for example, as SoftForm®); human adipose tissue; solubilized elastin peptides with bovine collagen (such as those marketed, for example, as Endoplast 50®); non-animal derived L-polylactic acid (such as those marketed, for example, as Sculptra®); dextran beads in hyalon gel (such as those marketed, for example, as Reviderm Intra®); siloxane polymers ranging from 1,000-5,000 centistoke (such as those marketed, for example, as Silikon® and/or AdatoSil®); and combinations thereof.

### Methods of Treatment

Compositions for the treatment of tissue contour defects or deformations are administered through intradermal injections at the desired site of treatment. Methods presented herein comprise selecting skin in need of tissue contour defect treatment and introducing into said skin compositions presented herein. Amounts to be injected can vary and may depend on the site of injection; exact amounts to achieve the desired tissue contour are readily determined by one of skill in the art. For instance, amounts may range from at least about 0.1 to at least about 15.0 cubic centimeters (cc) of volume. Even larger volumes as described herein may be used where the area of treatment requires or can accept larger volumes of solution. Areas treated with compositions of the present invention include, but are not limited to, the lips, perioral areas, the corners of the mouth, melolabial areas, forehead and glabella, perio-orbital and glabella, cheeks, as well as cheeks and lateral aspects of the jaw line. Further areas and contour defects or deformations suitable for treatment include scars at any location and of any type. Additional areas for treatment include any area where treatment or amelioration of skin contour deformation is desired. Such areas may be further identified by any person of skill in the art, such as a physician or dermatologist.

In one embodiment of the invention, the solution is injected into one compartment of the skin. In a further embodiment, the solution is injected into two compartments of the skin. In a further embodiment of the invention, the solution is injected into the reticular dermis and the papillary dermis. In yet a further embodiment, the solution is first injected into the reticular dermis followed by injection into the papillary dermis. In yet another embodiment the solution is injected into more than one compartment of the skin in a random order.

In an additional embodiment of the invention, the volume of solution injected is at least about 5 cubic centimeters. In a further embodiment, injection volumes include, but are not limited to, at least about 0.01, 0.5, 1.0, 1.5, 2.0, 3.0, 4.0, 5.0, 6.0, 7.0, 8.0, 9.0, 10.0, 11.0, 12.0, 13.0, 14.0, and at least about 15.0 cubic centimeters. In yet a further embodiment, the injection volume is greater than at least about 15.0 cubic centimeters, and include, but is not limited to, about 15.0 cubic centimeters to about 20 cubic centimeters, about 17.0 cubic centimeters to about 23.0 cubic centimeters, about 20 cubic centimeters to about 25 cubic centimeters, 22 cubic centimeters to about 28 cubic centimeters, 25 cubic centimeters to about 30 cubic centimeters, 15 cubic centimeters to about 30 cubic centimeters, or about 15 cubic centimeters to about 40 cubic centimeters.

In an embodiment of the invention described herein, including all aforementioned aspects and embodiments, the injectable composition is utilized for the treatment of wrinkles. The intradermal injection of the disclosed composition corrects the skin contour defects or deformations associated with type 1, type 2, type 3, and type 4 wrinkles. In additional embodiments, intradermal injections are used to correct scarring of the skin, including but not limited to surgical scars, trauma scars, and acne scarring. In yet another embodiment, intradermal injections of the compositions disclosed herein are used to correct and augment lip thinning as well as chin surface contour defects. Additionally, repeated injections on daily, weekly, monthly, or other time frames can correct skin contour defects or deformations as well as stimulate the regeneration of healthy skin, further alleviating and preventing skin contour defects or deformation.

Efficacy of intradermal injection of the disclosed compositions can be assessed by a variety of methods including: visual inspection of skin contour, length of efficacious period, physical assessment of dermal health, generation of new dermal growth, amount of collagen deposition in the dermis, and other assessment criteria known in the art. By way of example, physical assessment of the skin for renewed elasticity, thickness, and tone with no evidence of lumping, nodularity, shifting, or hypersensitivity is indicative of composition efficacy. Conversely, the absence of elasticity or thickness of the dermis, as with the absence of any other assessment criterion disclosed herein, is not necessarily indicative of a lack of efficacy. In the case of scar treatment with the compositions disclosed herein, changes in the contour and texture of scars can be indicative of composition efficacy.

Additional evaluation of cosmetic correction includes a validated Facial Fold Assessment Scale (FFA), utilizing standardized photographs or images as reference. FFA Scale ratings range from a “0” score of no folds to a “5” score of very deep folds with edges well-defined and some redundant folds. Assessment is performed by masked observers in a double-blind manner. Patient photos are taken at pre-treatment visits as well as at each follow-up visit, and efficacy is assessed for a twelve month period following completion of the treatments. Statistically significant improvement based on FFA is indicative of efficacious administration of hydrophilic polymer compositions disclosed herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

Dolsky R L., Cosmetic surgery in the United States: its past and present. Dermatol Surg. 1999 November; 25(11):886-92.
Pierard G E, Henry F, Castelli D, Ries G., Ageing and rheological properties of facial skin in women. Gerontology. 1998; 44(3):159-61.
Henry F, Pierard-Franchimont C, Cauwenbergh G, Pierard G E. Age-related changes in facial skin contours and rheology. J Am Geriatr Soc. 1997 February; 45(2):220-2.
Stegman, S J. Sleep creases. Am J Cosm Surg 1987; 4:277-280.
Fulton J E Jr, Gaminchi F. Sleep lines. Dermatol Surg. 1999 January; 25(1):59-62.
Pierard G E. EEMCO guidance to the in vivo assessment of tensile functional properties of the skin. Part 1: relevance to the structures and ageing of the skin and subcutaneous tissues. Skin Pharmacol Appl Skin Physiol. 1999 November-December; 12(6):352-62.
Sams W. Sun-induced aging. Dermatol Clin 1986; 4:509-16.
Gonzalez-Ullosa, M, Flores, E S. Senility of the face—Basic study to understand its causes and effects. Plast Reconstr Surg. 1965 August; 36:239-46. No abstract available.
Kligman A M, Zheng P, Lavker R M. The anatomy and pathogenesis of wrinkles. Br J Dermatol. 1985 July; 113(1):37-42.
Batisse D, Bazin R, Baldeweck T, Querleux B, Leveque J L. Influence of age on the wrinkling capacities of skin. Skin Res Technol. 2002 August; 8(3):148-54.
Setaro M, Abella M L, Sparavigna A, Leveque J L. Ptosis of submental skin: objective measurements and effect of age. Skin Res Technol. 2004 November; 10(4):251-6.
Bosset S, Barre P, Chalon A, Kurfurst R, Bonte F, Andre P, Perrier P, Disant F, Le Varlet B, Nicolas J F. Skin ageing: clinical and histopathologic study of permanent and reducible wrinkles. Eur J. Dermatol. 2002 May-June; 12(3):247-52.
Warren R, Gartstein V, Kligman A M, Montagna W, Allendorf R A, Ridder G M. Age, sunlight, and facial skin: a histologic and quantitative study. J Am Acad Dermatol. 1991 November; 25(5 Pt 1):751-60.
Pierard, G E, Uhoda I, Pierard-Franchimont C. From skin microrelief to wrinkles. An area ripe for investigation. Journal of Cosmetic Dermatology 2003 2:1 21
Contet-Audonneau J L, Jeanmaire C, Pauly G. A histological study of human wrinkle structures: comparison between sun-exposed areas of the face, with or without wrinkles, and sun-protected areas. Br J. Dermatol. 1999 June; 140(6):1038-47.
Bosset S, Bonnet-Duquennoy M, Barre P, Chalon A, Lazou K, Kurfurst R, Bonte F, Schnebert S, Disant F, Le Varlet B, Nicolas J F. Decreased expression of keratinocyte beta1 integrins in chronically sun-exposed skin in vivo. Br J. Dermatol. 2003 April; 148(4):770-8.
Greco M, Villani G, Mazzucchelli F, Bresolin N, Papa S, Attardi G. Marked aging-related decline in efficiency of oxidative phosphorylation in human skin fibroblasts. FASEB J. 2003 September; 17(12):1706-8. Epub 2003 Jul. 18.
Fulton J E Jr., Suarez M., Silverton K., Barnes T. Small volume fat transfer. Dermatol Surg. 1998 August; 24(8):857-65.
West M D. The cellular and molecular biology of skin aging. Arch Dermatol. 1994 January; 130(1):87-95.
Fisher G J. The pathophysiology of photoaging of the skin. Cutis. 2005 February; 75(2 Suppl):5-8; discussion 8-9. Watson R E, Ball S G, Craven N M, Boorsma J, East C L, Shuttleworth C A, Kielty C M, Griffiths C E. Distribution and expression of type VI collagen in photoaged skin. Br J. Dermatol. 2001 April; 144(4):751-9.
Watson R E, Griffiths C E, Craven N M, Shuttleworth C A, Kielty C M. Fibrillin-rich microfibrils are reduced in photoaged skin. Distribution at the dermal-epidermal junction. J Invest Dermatol. 1999 May; 112(5):782-7.
Kiss M, Wlaschek M, Brenneisen P, Michel G, Hommel C, Lange T S, Peus D, Kemeny L, Dobozy A, Scharffetter-Kochanek K, et al. Alpha-melanocyte stimulating hormone induces collagenase/matrix metalloproteinase-1 in human dermal fibroblasts. Biol Chem Hoppe Seyler. 1995 July; 376(7):425-30.
Ryan T. The ageing of the blood supply and the lymphatic drainage of the skin. Micron. 2004; 35(3):161-71.
Monheit G D, Chastain M A. Chemical peels. Facial Plast Surg Clin North Am 2001; 9:239-55.
Bernstein E F, Andersen D, Zelickson B D. Laser resurfacing for dermal photoaging. Clin Plast Surg 2000; 27:221-40. Fulton J E Jr., Barnes T. Collagen shrinkage (selective dermaplasty) with the high-energy pulsed carbon dioxide laser. Dermatol Surg. 1998 January; 24(1):37-41.
Bitter P H. Noninvasive rejuvenation of photodamaged skin using serial, full-face intense pulsed light treatments. Dermatol Surg. 2000 September; 26(9):835-42.
Fligiel Suzanne E G, James Varani, Subhash C. Datta, Sewon Kang, Gary J. Fisher, John J. Voorhees. Collagen Degradation in Aged/Photodamaged Skin In Vivo and After Exposure to Matrix Metalloproteinase-1 In Vitro. Journal of investigative Dermatology 2003 120:5 842
Varani James, Lucia Schuger, Michael K. Dame, Christina Leonard, Suzanne E. G. Fligiel, Sewon Kang, Gary J. Fisher, John J. Voorhees. Reduced Fibroblast Interaction with Intact Collagen as a Mechanism for Depressed Collagen Synthesis in Photodamaged Skin. Journal of Investigative Dermatology 2004 122:6 1471
Craven N M, Watson R E, Jones C J, Shuttleworth C A, Kielty C M, Griffiths C E. Clinical features of photodamaged human skin are associated with a reduction in collagen VII. Br J. Dermatol. 1997 September; 137(3):344-50.
Weiss R A, Goldman M P, Weiss M A. Treatment of poikiloderma of Civatte with an intense pulsed light source. Dermatol Surg. 2000 September; 26(9):823-7; discussion 828.
Jin Ho Chung, Jin Young Seo, Hai Ryung Choi, Mi Kyung Lee, Choon Shik Youn, Gi-eun Rhie, Kwang Hyun Cho, Kyu Han Kim, Kyung Chan Park, Hee Chul Eun. Journal of Investigative Dermatology 2001 117:5 1218
El-Domyati M, Attia S, Saleh F, Brown D, Birk D E, Gasparro F, Ahmad H, Uitto J. Intrinsic aging vs. photoaging: a comparative histopathological, immunohistochemical, and ultrastructural study of skin. Exp Dermatol. 2002 October; 11(5):398-405.
Kligman A M, Dogadkina D, Lavker R M. Effects of topical tretinoin on non-sun-exposed protected skin of the elderly. J Am Acad Dermatol. 1993 July; 29(1):25-33.
Kligman A M, Leyden J J. Treatment of photoaged skin with topical tretinoin. Skin Pharmacol. 1993; 6 Suppl 1:78-82.
Bernstein E F, Underhill C B, Lakkakorpi J, Ditre C M, Uitto J, Yu R J, Scott E V. Citric acid increases viable epidermal thickness and glycosaminoglycan content of sun-damaged skin. Dermatol Surg. 1997 August; 23(8):689-94.
Watson R E, Craven N M, Kang S, Jones C J, Kielty C M, Griffiths C E. A short-term screening protocol, using fibrillin-1 as a reporter molecule, for photoaging repair agents. J Invest Dermatol. 2001 May; 116(5):672-8.
Rubino C, Farace F, Dessy L A, Sanna M P, Mazzarello V. A prospective study of anti-aging topical therapies using a quantitative method of assessment.
Plast Reconstr Surg. 2005 April; 115(4):1156-62; discussion 1163-4. Coimbra M, Rohrich R J, Chao J, Brown S A. A prospective controlled assessment of microdermabrasion for damaged skin and fine rhytides.
Plast Reconstr Surg. 2004 Apr. 15; 113(5):1438-43; discussion 1444.
Brody H J. Histology and classification In: Brody H G ed. Chemical Peeling and Resurfacing. 2^{nd} ed. St. Louis; Mosby yearbook, 1997:1-6.
Brody H J. Complications of chemical resurfacing. Dermatol Clin. 2001 July; 19(3):427-38, vii-viii.
Ross E V, Miller C, Meehan K, Pac, McKinlay J, Sajben P, Trafeli J P, Barnette D J. One-pass CO2 versus multiple-pass Er:YAG laser resurfacing in the treatment of rhytides: a comparison side-by-side study of pulsed CO2 and Er:YAG lasers. Dermatol Surg. 2001 August; 27(8):709-15.
Goldman M P, Marchell N, Fitzpatrick R E. Laser skin resurfacing of the face with a combined CO2/Er:YAG laser. Dermatol Surg. 2000 February; 26(2): 102-4.
Fitzpatrick R E, Goldman M P, Sriprachya-Anunt S. Resurfacing of photodamaged skin on the neck with an UltraPulse® carbon dioxide laser. Lasers Surg Med. 2001; 28(2):145-9.
Roenigk R K, Brodland M P, Satur N M, Tope W D. A primer of facial chemical peel. Dermatol Clin 1993; 11:349-59.
Goldberg D J, Silapunt S. Q-switched Nd:YAG laser: rhytid improvement by non-ablative dermal remodeling. J Cutan Laser Ther. 2000 September; 2(3):157-60.
Rhesa May P. Manaloto M D & Tina S. Alster M D. Periorbital rejuvenation: A review of dermatologic treatments. Dermatol Surg. 1999 January; 25 (1): 1-9.
Fitzpatrick R E, Goldman M P, Satur N M, Tope W D. Pulsed carbon dioxide laser resurfacing of photo-aged facial skin. Arch Dermatol. 1996 April; 132(4):395-402. Fagien S, Elson M L. Facial soft-tissue augmentation with allogeneic human tissue collagen matrix (Dermalogen and Dermaplant). Clin Plast Surg. 2001 January; 28(1):63-81.
Alstair T S. Laser resurfacing of rhytides. In: Alstaer T S. Manual of Cutaneous Laser Techniques. Philadelphia: Lippincott-Raven Publishers, 1997; 104-22. Rohrich R J, Reagan B J, Adarns W P Jr, Kenkel J M, Beran S J. Early results of vermilion lip augmentation using acellular allogeneic dermis: an adjunct in facial rejuvenation. Reconstr Surg. 2000 January; 105(1):409-16; discussion 417-8.
Ruiz-Esparza J, Barba Gomez J M, Gomez de la Torre O L, David L. Erythema after laser skin resurfacing. Dermatol Surg. 1998 January; 24(1):31-4.
Rapaport, M J, Rapaport, V. Prolonged erythema after facial laser resurfacing or phenol peel. Dermatol Surg 1999 October; 25(10):781-785.
Ang P, Barlow R J. Nonablative laser resurfacing: a systematic review of the literature. Clin Exp Dermatol. 2002 November; 27(8):630-5.
Weiss R A, Weiss M A, Beasley K L. Rejuvenation of photoaged skin: 5 years results with intense pulsed light of the face, neck, and chest. Dermatol Surg. 2002 December; 28(12):1115-9.
Goldberg D J. New collagen formation after dermal remodeling with an intense pulsed light source. J Cutan Laser Ther. 2000 June; 2(2):59-61.
Goldberg D J. Full-face nonablative dermal remodeling with a 1320 nm Nd:YAG laser. Dermatol Surg 2001 August; 27(8):781-2.
Weiss R A, Weiss M A, Geronemus R G, McDaniel D H. A novel non-thermal non-ablative full panel LED photomodulation device for reversal of photoaging: digital microscopic and clinical results in various skin types. J Drugs Dermatol. 2004 November-December; 3(6):605-10.
Leffell D J. Clinical efficacy of devices for nonablative photorejuvenation: Arch Dermatol. 2002 November; 138(11):1503-8.
Papadavid E, Katsambas A. Lasers for facial rejuvenation: a review.
Int J. Dermatol. 2003 June; 42(6):480-7.
Carruthers J D A, Carruthers A, The Treatment of Glabellar Furrows with Botulinum-A Exotoxin. J. Dermatol Surg Oncol, 1990; 16; 83
Carruthers J D A, Carruthers A. “Treatment of glabellar frown Lines with C. botulinum A Exotoxin. J. Dermatol Surg Oncol 1992; 18:17-21—Abstracted in International Abstracts Dermatology April/May/June 1992, pg. 7
Carruthers A and Carruthers J. The use of botulinum toxin to treat glabellar frown lines and other facial wrinkles. Cosmetic Dermatology, Vol. 7, No. 5, May 1994, pp 12-15
Wider J, Moy R. Understanding botulinum toxin, surgery anatomy of the frown, forehead, and periocular region. Dermatol Surg 1998; 24:1172-4.
West T B, Alster T S. Effect of botulinum toxin type A on movement-associated rhytides following CO2 laser resurfacing. Dermatol Surg. 1999 April; 25(4):259-61.
Carruthers J, Carruthers A. A prospective, randomized, parallel group study analyzing the effect of BTX-A (Botox) and nonanimal sourced hyaluronic acid (NASHA, Restylane) in combination compared with NASHA (Restylane) alone in severe glabellar rhytides in adult female subjects: treatment of severe glabellar rhytides with a hyaluronic acid derivative compared with the derivative and BTX-A. Dermatol Surg. 2003 August; 29(8):802-9.
Schanz S, Schippert W, Ulmer A, Rassner G, Fierlbeck G. Arterial embolization caused by injection of hyaluronic acid (Restylane). Br. J. Dermatol. 2002 May; 146(5):928-9.
Guyuron B, Rose K, Kriegler J S, Tucker T. Hourglass deformity after botulinum toxin type A injection. Headache. 2004 March; 44(3):262-4.
Donofrio L M. Structural autologous lipoaugmentation: a pan-facial technique.
Dermatol Surg. 2000 December; 26(12):1129-34.
Sclafani A P, Romo T 3rd. Injectable fillers for facial soft tissue enhancement. Facial Plast Surg. 2000; 16(1): 29-34.
Elson M L. Soft tissue augmentation. A review.
Dermatol Surg. 1995 June; 21(6):491-500; quiz 501-2.
Cheng J T, Perkins S W, Hamilton M M. Collagen and injectable fillers. Otolaryngol Clin North Am. 2002 February; 35(1):73-85, vi.
Sclafani A P, Romo T 3rd. Collagen, human collagen, and fat: the search for a three-dimensional soft tissue filler. Facial Plast Surg. 2001 February; 17(1):79-85.
Homicz M R, Watson D. Review of injectable materials for soft tissue augmentation. Facial Plast Surg. 2004 February; 20(1):21-9. Markey A C, Glogau R G. Autologous fat grafting: comparison of techniques. Dermatol Surg. 2000 December; 26(12): 1135-9.
Biesman B. Soft tissue augmentation using Restylane. Facial Plast Surg. 2004 May; 20(2):171-7; discussion 178-9.
Dover J S, Carruthers A, Carruthers J, Alam M. Clinical Use of RESTYLANE. Skin Therapy Lett. 2005 February; 10(1):5-7.
Carruthers J, Klein A W, Carruthers A, Glogau R G, Canfield D. Safety and efficacy of nonanimal stabilized hyaluronic acid for improvement of mouth corners. Dermatol Surg. 2005 March; 31(3):276-80.
Soparkar C N, Patrinely J R. Managing inflammatory reaction to restylane. Ophthal Plast Reconstr Surg. 2005 March; 21(2):151-3.
Andre P. Evaluation of the safety of a non-animal stabilized hyaluronic acid (NASHA—Q-Medical, Sweden) in European countries: a retrospective study from 1997 to 2001. J Eur Acad Dermatol Venereol. 2004 July; 18(4):422-5.
Friedman P M, Mafong E A, Kauvar A N, Geronemus R G. Safety data of injectable nonanimal stabilized hyaluronic acid gel for soft tissue augmentation. Dermatol Surg. 2002 June; 28(6):491-4.
Lowe N J, Maxwell C A, Lowe P, Duick M G, Shah K. Hyaluronic acid skin fillers: adverse reactions and skin testing. J Am Acad Dermatol. 2001 December; 45(6):930-3.
Fernandez-Acenero M J, Zamora E, Borbujo J. Granulomatous foreign body reaction against hyaluronic acid: report of a case after lip augmentation. Dermatol Surg. 2003 December; 29(12):1225-6.
Donofrio L M. Fat distribution: a morphologic study of the aging face. Dermatol Surg. 2000 December; 26(12):110 Donofrio L. Technique of periorbital lipoaugmentation. Dermatol Surg. 2003 January; 29(1):92-8.
Markey A C, Glogau R G. Autologous fat grafting: comparison of techniques. Dermatol Surg. 2000 December; 26(12): 1135-9.
Fagien S. Facial soft-tissue augmentation with injectable autologous and allogeneic human tissue collagen matrix (autologen and dermalogen). Reconstr Surg. 2000 January; 105(1):362-73; discussion 374-5.
Sclafani A P, Romo T 3rd, Parker A, McCormick SA, Cocker R, Jacono A. Homologous collagen dispersion (dermalogen) as a dermal filler: persistence and histology compared with bovine collagen. Ann Plast Surg. 2002 August; 49(2):181-8.
Sclafani A P, Romo T 3rd, Jacono A A. Rejuvenation of the aging lip with an injectable acellular dermal graft (Cymetra). Arch Facial Plast Surg. 2002 October-December; 4(4):252-7.
Bauman L. CosmoDerm/CosmoPlast (human bioengineered collagen) for the aging face. Facial Plast Surg. 2004 May; 20(2): 125-8.
Cohen S R, Holmes R E. Artecoll: a long-lasting injectable wrinkle filler material: Report of a controlled, randomized, multicenter clinical trial of 251 subjects. Plast Reconstr Surg. 2004 September 15; 114(4):964-76; discussion 977-9.
Lemperle G, Gauthier-Hazan N, Lemperle M. PMMA-Microspheres (Artecoll) for long-lasting correction of wrinkles: refinements and statistical results. Aesthetic Plast Surg. 1998 September-October; 22(5):356-65.
Alcalay J, Alkalay R, Gat A, Yorav S. Late-onset granulomatous reaction to Artecoll. Dermatol Surg. 2003 August; 29(8):859-62.
Kim K J, Lee H W, Lee M W, Choi J H, Moon K C, Koh J K. Artecoll granuloma: a rare adverse reaction induced by microimplant in the treatment of neck wrinkles. Dermatol Surg. 2004 April; 30(4 Pt 1):545-7.
Fulton J E Jr, Rahimi A D, Helton P, Watson T, Dahlberg K. Lip rejuvenation. Dermatol Surg. 2000 May; 26(5):470-4; discussion 474-5.
Leveque J L, Goubanova E. Influence of age on the lips and perioral skin. Dermatology. 2004; 208(4):307-13.
Bisson M, Grobbelaar A. The esthetic properties of lips: a comparison of models and nonmodels. Angle Orthod. 2004 April; 74(2):162-6.
Fagien S. Facial soft-tissue augmentation with injectable autologous and allogeneic human tissue collagen matrix (autologen and dermalogen). Plast Reconstr Surg. 2000 January; 105(1):362-73; discussion 374-5.
Duffy D M. Complications of fillers: an overview. Dermatol Surg 2005; 31:1626-1633.
Beare-Rogers J et al. Pure and applied chemistry 73, 2001, 685-744.
Theis, J G, Liau-Chu M, Chan, H S, Doyle J, Greenberg M L, Koren G. Anaphylactoid reactions in children receiving high-dose intravenous cyclosporine for reversal of tumor resistance: the causative role of improper dissolution of Cremophor E L. J Clin Oncol. 1995 October; 13(10):2508-16.

What is claimed is:

1. A pharmaceutically acceptable, injectable composition for the treatment of skin contour defects or deformations comprising at least one ethoxylated castor oil and a physiologically acceptable solvent.

2. The composition of claim 1, wherein the ethoxylated castor oil comprises a polyoxyethylene glycol group.

3. The composition of claim 1, wherein the ethoxylated castor oil has been obtained by the reaction of castor oil and ethylene oxide in a ratio of between about 1:30 and about 1:45.

4. The composition of claim 1, wherein the ethoxylated castor oil is PEG-35 castor oil.

5. The composition of claim 1, comprising from about 0.1% to about 40.0% w/w of the ethoxylated castor oil.

6. The composition of claim 5, comprising about 40% w/w of PEG-35 castor oil.

7. The composition of claim 1, further comprising at least one anesthetic, preservative, tonicity agent, or combinations thereof.

8. The composition of claim 7, wherein the anesthetic is lidocaine.

9. A method of treating skin contour defects or deformations comprising:
injecting a pharmaceutically acceptable composition comprising at least one ethoxylated castor oil and
a physiologically acceptable solvent to at least one compartment of a patient's skin;
wherein the skin surface exhibits at least one contour defect or deformation; and
whereby the skin contour defect or deformation is treated after the injection.

10. The method of claim 9, wherein the ethoxylated castor oil in the composition comprises a polyoxyethylene glycol group.

11. The method of claim 9, wherein the ethoxylated castor oil in the composition has been obtained by the reaction of castor oil and ethylene oxide in a ratio of between about 1:30 and about 1:45.

12. The method of claim 9, wherein the ethoxylated castor oil in the composition is PEG-35 castor oil.

13. The method of claim 1, wherein the composition comprises from about 0.1% to about 40.0% w/w of the ethoxylated castor oil.

14. The method of claim 13, wherein the composition comprises about 40% w/w PEG-35 castor oil.

15. The method of claim 9, wherein the composition further comprises at least one anesthetic, preservative, tonicity agent, or combinations thereof.

16. The method of claim 15, wherein the anesthetic in the composition is lidocaine.

17. The method of claim 9, wherein the composition is first injected into the reticular dermis, and then injected into the papillary dermis.

18. The method of claim 9, wherein the volume of the composition ranges from about 0.01 to about 60.0 cubic centimeters.

19. The method of claim 18, wherein the volume of the composition ranges from about 5.0 to about 40.0 cubic centimeters.

20. The method of claim 9, wherein the skin contour defect comprises at least one wrinkle, scar, lip thinning, or combinations thereof.

21. A method of manufacturing a pharmaceutically acceptable, injectable composition for the treatment of skin contour defects or deformations comprising:
a. solubilizing at least one ethoxylated castor oil in a physiologically acceptable solvent;
b. sterilizing the solution comprising at least one ethoxylated castor oil; and
c. mixing the solution comprising at least one ethoxylated castor oil.

22. The method of claim 21, further comprising adding at least one anesthetic, preservative, tonicity agent, or combinations thereof after sterilizing the solution.
